(19) United States
(12) Patent Application Publication
(10) Pub. No.: US 2026/0093048 A1
(43) Pub. Date: Apr. 2, 2026

(54) RADIATION DETECTOR MODULE, RADIATION DETECTOR AND MANUFACTURING METHOD THEREFOR, AND IMAGING DEVICE

(71) Applicant: GE Precision Healthcare LLC, Waukesha, WI (US)

(72) Inventors: Qinglei Li, Beijing (CN); Yuwei Zhang, Beijing (CN); Zhiqiang Liu, Beijing (CN); Dongmei Wang, Beijing (CN)

(21) Appl. No.: 19/340,693

(22) Filed: Sep. 25, 2025

(30) Foreign Application Priority Data
Sep. 27, 2024 (CN) 202411370039.2
Sep. 27, 2024 (CN) 202422383338.1

(51) Int. Cl. G01T 1/20; A61B 6/03; A61B 6/42

(52) U.S. Cl. CPC G01T 1/20184; A61B 6/032; A61B 6/4233; G01T 1/2002

(57) ABSTRACT

Embodiments of the present application provide a radiation detector module, a radiation detector, and an imaging device. In the radiation detector module, the number of radiation detector elements arranged in the Z direction of a circuit substrate is 4N rows or 4N+1 rows, N being an integer greater than or equal to 1. In this way, the radiation detector elements can be removed in a symmetric manner with respect to a center of the circuit substrate in the Z direction, for example, 2N rows of radiation detector elements are removed, such that the central position of the remaining radiation detector elements in the Z direction remains unchanged. Therefore, neither the positions of the remaining radiation detector elements nor the focus position of an X-ray source requires adjustment. Thus, no change of a gantry structure is required, and additional mechanical vibration is not caused, thereby ensuring imaging quality.

### TECHNICAL FIELD

This application claims priority to Chinese Application No. 202411370039.2, filed on September 27, 2024, and claims priority to Chinese Application No. 202422383338.1, filed on September 27, 2024, the disclosures of which are incorporated herein by reference in their entirety.

### TECHNICAL FIELD

Embodiments of the present application relate to the technical field of imaging devices, and in particular, to a radiation detector module, a radiation detector and a manufacturing method therefor, and an imaging device.

### BACKGROUND

Imaging devices are used to scan a subject under examination (such as a patient or a workpiece) in a non-invasive or non-destructive manner, to obtain an internal structure image of an anatomical tissue or site of interest of the subject under examination to assist in diagnosis.

Imaging devices usually include a circular scanning bore (for example, a scanning gantry opening) for a subject under scanning examination to move in or out, and include a detector subsystem mounted along the entire circumference or a partial arc of the circular bore. The detector subsystem includes a plurality of detector modules mounted on a gantry. For example, a computed tomography (CT) device is generally used as a medical imaging device for scanning a patient to acquire a tomographic medical image of a site of interest of the patient, so as to assist a doctor in diagnosis.

A CT device includes a plurality of radiation detector modules that receive X-rays emitted from an X-ray tube and passing through the patient, and the form and number of the radiation detector modules depend on clinical needs and the design of a CT system. Communication connections are established between the radiation detector modules. Each radiation detector module of the CT device generally includes a radiation detector element. The radiation detector element may include, for example, a pixelated scintillator and a photoelectric conversion component sequentially arranged along a ray transmission direction, the scintillator being used to receive the X-rays passing through the patient and generate light, and the photoelectric conversion component (such as a photodiode) converting the light generated by the scintillator into an electrical signal. Each detector module further includes a collimator, used to collimate the X-rays passing through the patient to a specific direction, so as to prevent or reduce interference between pixels of the scintillator. Each detector module further includes a signal processing circuit, used to process the electrical signal generated by the photoelectric conversion component, and a frame for supporting the collimator, the scintillator, the photoelectric conversion component, a circuit board, and a heat dissipation component.

The CT device further includes a computer subsystem to reconstruct, based on processed electrical signals, to generate a medical tomographic image for assisting in diagnosis.

It should be noted that the above introduction of the background is only for the convenience of clearly and completely describing the technical solutions of the present application, and for the convenience of understanding for those skilled in the art.

### SUMMARY OF THE INVENTION

In a CT device, the number of images formed by one exposure of a radiation detector module depends on the number of radiation detector elements arranged in the radiation detector module in the Z direction (i.e., a direction in which a subject under examination enters or exits the CT device from a scanning hole) and the number of ray transmission channels of each radiation detector element in the Z direction. Generally, a higher number of radiation detector elements arranged in the Z direction indicates a shorter examination time, which is more suitable for the examination of a body part that is more movable. In some use scenarios (for example, when a relatively stationary body part is examined), the number of radiation detector elements arranged in the Z direction may be lower, thereby saving costs.

The inventors have found that in an imaging device such as a CT device, if the number of radiation detector elements in the Z direction in the radiation detector module is reduced (for example, reduced by half), the central position of the remaining radiation detector elements in the Z direction may be shifted. Therefore, it is necessary to adjust the position of the focus of an X-ray source in the Z direction, or adjust the positions of the remaining radiation detector elements in the Z direction, to cause the focus of the X-ray source to be located at the central position of the remaining radiation detector elements in the Z direction, and such adjustment of the focus position of the X-ray source may affect a gantry structure design or mechanical vibration of the imaging device, thus affecting imaging quality.

In view of at least one of the above technical problems, or other similar problems, embodiments of the present application provide a radiation detector module, a radiation detector and a manufacturing method therefor, and an imaging device. In the radiation detector module, the number of radiation detector elements arranged in the Z direction of a circuit substrate is 4N rows or 4N+1 rows, N being an integer greater than or equal to 1. In this way, in a scenario requiring removal of a portion of the radiation detector elements, the radiation detector elements can be removed in a symmetric manner with respect to a center of the circuit substrate in the Z direction, for example, 2N rows of radiation detector elements are removed, such that the central position of the remaining radiation detector elements in the Z direction remains unchanged. Therefore, neither the positions of the remaining radiation detector elements nor the focus position of a radiation source (for example, an X-ray source) requires adjustment. Thus, no change of a gantry structure is required, and additional mechanical vibration is not caused, thereby ensuring imaging quality.

According to an aspect of the embodiments of the present application, a radiation detector module is provided. The radiation detector module is used to detect a ray signal passing through a subject under examination in an imaging device, the subject under examination entering or exiting the imaging device in a first direction. The radiation detector module includes a radiation detector element, receiving a ray emitted by a radiation source and converting the ray into an electrical signal; a circuit substrate, a plurality of radiation detector elements being mounted at a first side of the circuit substrate, and the plurality of radiation detector elements being arranged in 4N rows or 4N+1 rows in a first direction of the circuit substrate, N being an integer greater than or equal to 1; and a processing circuit chip, disposed at a second side of the substrate and communicating with the radiation detector elements.

In this way, the radiation detector can not only support imaging an image of a specific number of rows, but also directly remove 2N rows of radiation detector elements and related assemblies that are arranged on the circuit substrate in the Z direction in a centrally symmetric manner, to support imaging an image of about half of the number of rows. In addition, the central position of the radiation detector in the Z direction remains unchanged, so that the structures of a detector, a gantry, and other components or devices require no change, thereby reducing manufacturing costs.

In some embodiments, in the first direction, the plurality of radiation detector elements are arranged symmetrically with respect to the central position of the circuit substrate in the first direction. In some embodiments, each of the radiation detector elements has 16 rows of ray transmission channels distributed along the first direction. In some embodiments, the plurality of radiation detector elements are arranged in one column or two or in more columns in a second direction of the circuit substrate. In some embodiments, one or a plurality of processing circuit chips is/are disposed in a region of the circuit substrate covered by each radiation detector element. In some embodiments, the radiation detector element is electrically connected to the processing circuit chip by means of a conductive path penetrating through the circuit substrate.

In some embodiments, the radiation detector element comprises a scintillator and a photoelectric conversion element, the scintillator receives a ray and generates light, the photoelectric conversion element converts the light generated by the scintillator into an electrical signal, and the photoelectric conversion element comprises a backlit photodiode. In some embodiments, the radiation detector module has a flat panel form factor. In some embodiments, the radiation detector module further includes a data collection circuit board, electrically connected to the circuit substrate by means of a wire, and receiving data processed by the processing circuit chip.

In some embodiments, the radiation detector module further includes a collimator assembly, disposed on a surface of the radiation detector component, the collimator assembly collimating a ray emitted by the radiation source to the radiation detector element.

According to another aspect of embodiments of the present application, a radiation detector is provided. The radiation detector includes a guide rail and two or more radiation detector modules according to any one of the above embodiments supported on the guide rail.

According to yet another aspect of the embodiments of the present application, an imaging device is provided. The imaging device comprises a scanning space for accommodating a subject under examination, the subject under examination entering or exiting the scanning space in a first direction, the imaging device comprises the radiation detector described in the above embodiments and an image reconstruction apparatus, and the image reconstruction apparatus performs image reconstruction based on an electrical signal generated by a radiation detector element in a radiation detector module of the radiation detector, so as to generate a tomographic image of the subject under examination.

According to another aspect of the embodiments of the present application, a manufacturing method for a radiation detector is provided. The manufacturing method of a radiation detector includes mounting two or more radiation detector modules according to any one of the above embodiments on a guide rail. In some embodiments, the two or more radiation detector modules are arranged along an extension direction of the guide rail.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT device according to an embodiment of the present application;

FIG. 2 is a schematic diagram of a CT imaging system according to an embodiment of the present application;

FIG. 3 is a schematic diagram of a cross section of a radiation detector module for comparison;

FIG. 4 is a schematic top view of a radiation detector module for comparison;

FIG. 5 is a schematic diagram of a cross section of a radiation detector module after a portion of the radiation detector elements in FIG. 3 are removed;

FIG. 6 is a top view of FIG. 5;

FIG. 7 is another schematic diagram of a cross section of a radiation detector module after a portion of the radiation detector elements in FIG. 3 are removed;

FIG. 8 is a top view of FIG. 7;

FIG. 9 is a schematic diagram of a cross section of a radiation detector module according to an embodiment of the present application;

FIG. 10 is a schematic top view of a radiation detector module according to an embodiment of the present application;

FIG. 11 is a schematic diagram of FIG. 9 with a portion of the radiation detector elements removed;

FIG. 12 is a schematic diagram of FIG. 10 with a portion of the radiation detector elements removed;

FIG. 13 is another top view of a radiation detector module according to an embodiment of the present application;

FIG. 14 is a schematic diagram of FIG. 13 with a portion of the radiation detector elements removed;

FIG. 15 is another schematic diagram of FIG. 13 with a portion of the radiation detector elements removed;

FIG. 16 is a three-dimensional schematic diagram of a radiation detector according to an embodiment of the present application;

FIG. 17 is a schematic diagram of a composition of an imaging device; and

FIG. 18 shows a manufacturing method for a radiation detector according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. In addition, the term “the” should be construed as including both the singular and plural forms, unless otherwise specified in the context. In addition, the term “according to” should be construed as “at least partially according to ...” and the term “based on” should be construed as “at least partially based on ...”, unless otherwise explicitly specified in the context.

The features described and/or illustrated for one implementation may be used in one or more other implementations in the same or similar way, be combined with features in other implementations, or replace features in other implementations. The terms “include/comprise” when used herein refer to the presence of features, integrated components, steps, or assemblies, but do not preclude the presence or addition of one or more other features, integrated components, steps, or assemblies.

In the embodiments of the present application, “or more” and “or less” include the number itself. For example, two or more includes two and more than two, and two or less includes two and less than two.

The medical imaging device described in the present application is applicable to various medical imaging modalities. The medical imaging device includes, but is not limited to, a computed tomography (CT) imaging device, or a positron emission tomography (PET) CT, or any other suitable medical imaging device.

The system obtaining the medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud. For example, the system obtaining the medical image data may be a CT imaging system, etc.

As an example, the embodiments of the present application are described below in conjunction with an X-ray computed tomography (CT) imaging device. Those skilled in the art would appreciate that the embodiments of the present application can also be applied to other medical imaging devices.

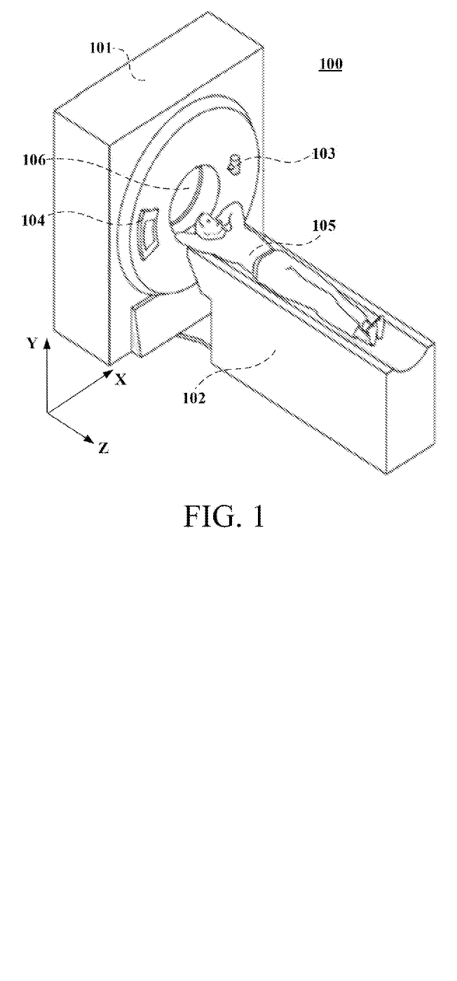

In embodiments of the present application the X direction is, for example, a direction in which each point of an arc shown in FIG. 1 points to an X-ray source 103, and the X direction is the transverse or left-right direction of a scanning gantry 101 or a patient table 102 shown in FIG. 1. The Y direction is, for example, a tangential direction of an arc centered on the X-ray source 103 shown in FIG. 1, the arc may represent, for example, an extension trajectory of a guide rail 1201 described later, the Y direction is the up-down direction of the scanning gantry 101 or the patient table 102 shown in FIG. 1, and the Y direction may also be referred to as a second direction. The Z direction is, for example, a direction in which the patient table 102 shown in FIG. 1 is moved in and out with respect to a scanning gantry opening 106, the Z direction is the front-rear direction of the scanning gantry 101 and the scanning gantry opening 106 shown in FIG. 1, and the Z direction may also be referred to as a first direction.

FIG. 1 is a schematic diagram of a CT device according to an embodiment of the present application, and schematically shows a CT device 100. As shown in FIG. 1, the CT device 100 includes a scanning gantry 101 and a patient table 102. The scanning gantry 101 has an X-ray source 103, and the X-ray source 103 projects an X-ray beam toward a detector assembly or collimator 104 on an opposite side of the scanning gantry 101. A subject under examination 105 may lie flat on the patient table 102 and be moved into a scanning gantry opening 106 along with the patient table 102. Medical image data of the subject under examination 105 may be obtained by means of scanning performed by the X-ray source 103.

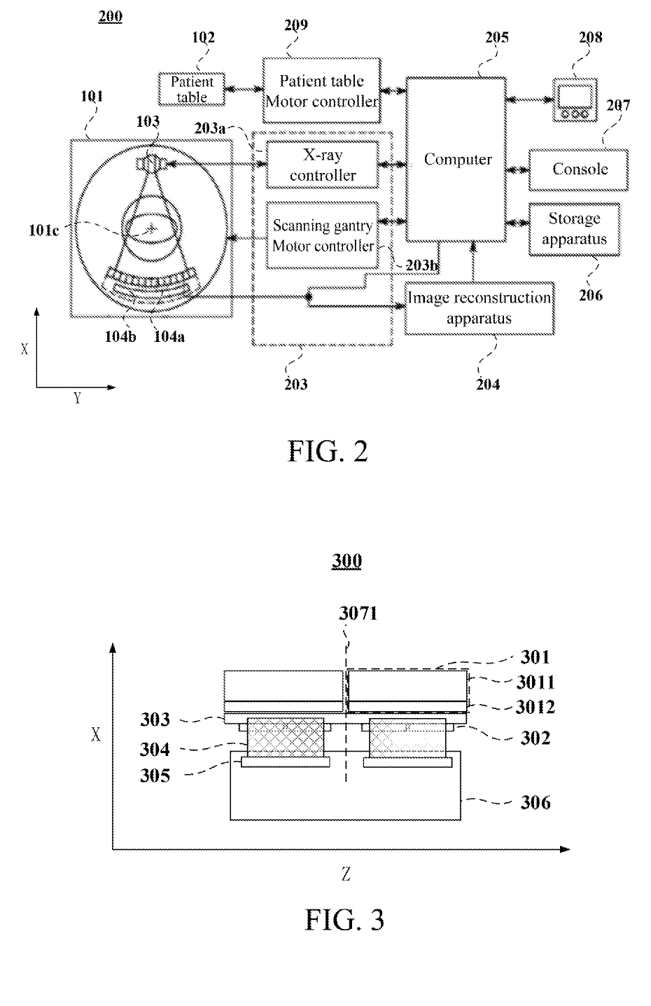

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through a subject under examination 105.

The DAS 104b, based on sensing of the detector units 104a, converts collected information into projection data for subsequent processing. During the scanning for acquiring the X-ray projection data, the scanning gantry 101 and components mounted thereon rotate around a center of rotation 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103, and a scanning gantry motor controller 203b that controls the rotational speed and position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface in a certain form, such as a keyboard, a mouse, a voice activated controller, or any other suitable input apparatus. An associated display 208 allows the operator to observe a reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102, so as to position the subject under examination 105 and the scanning gantry 101. In particular, the patient table 102 moves the subject under examination 105 to fully or partially pass through the scanning gantry opening 106 in FIG. 1.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. The storage device may be located in the medical imaging device, in a server outside the medical imaging device, in an independent medical imaging storage system (such as a picture archiving and communication system (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical image scanning is schematically illustrated above, and the embodiments of the present application are described in detail below with reference to the drawings. In the embodiments described below, the imaging device being a CT device is used as an example for description, and the content of the description is also applicable to other medical imaging devices.

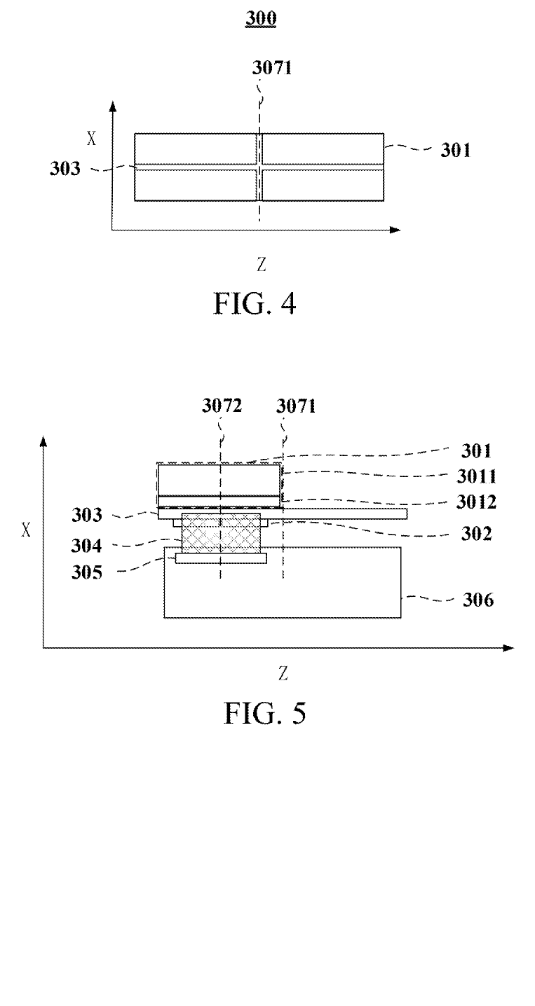

FIG. 3 is a schematic diagram of a cross section of a radiation detector module for comparison, viewed in the Y direction. FIG. 4 is a schematic top view of a radiation detector module for comparison, viewed in the X direction. As shown in FIG. 3 and FIG. 4, in the comparison, the radiation detector module 300 is used to detect a ray signal passing through a subject under examination 105 (shown in FIG. 1) in an imaging device, and the subject under examination 105 enters or exits the imaging device in the Z direction.

The radiation detector module 300 includes a radiation detector element 301, a processing circuit chip 302, and a circuit substrate 303. The radiation detector element 301 receives a ray emitted by a radiation source (for example, the X-ray source 103 shown in FIG. 1) and converts the ray into an electrical signal. A plurality of radiation detector elements 301 are mounted at a first side of the circuit substrate 303 (for example, the side facing the radiation source in the X direction). The plurality of radiation detector elements 301 are arranged in two rows in the Z direction of the circuit substrate 303. A center of the radiation detector module 300 along the Z direction (for example, a geometric center of the whole formed by the plurality of radiation detector elements 301) is located at the position of a dashed line 3071, for example, a focusing position of a ray projected by the radiation source to the radiation detector module 300. The processing circuit chip 302 is disposed at a second side of the circuit substrate 303 (for example, the side away from the radiation source in the X direction), and communicates with the radiation detector element 301.

In some examples, the radiation detector element 301 includes a scintillator 3011 and a photoelectric conversion element 3012. The radiation detector module 300 further includes a data collection circuit board 306, a flexible circuit 304, and a connection circuit 305. The radiation detector module 300 of FIG. 3 and FIG. 4 can, for example, acquire images of 64 rows of channels, and if it is required to refit the radiation detector module to acquire images of 32 rows of channels, the number of radiation detector components 301 distributed in the Z direction may be reduced by half, for example, the number of radiation detector elements 301 arranged in the Z direction can be reduced from 2 rows to 1 row. To keep the focus of the radiation source at the central position of the remaining radiation detector elements 301 in the Z direction, the position of the focus of the X-ray source in the Z direction may be adjusted, or the positions of the remaining radiation detector elements in the Z direction may be adjusted, as shown in FIG. 5 to FIG. 8.

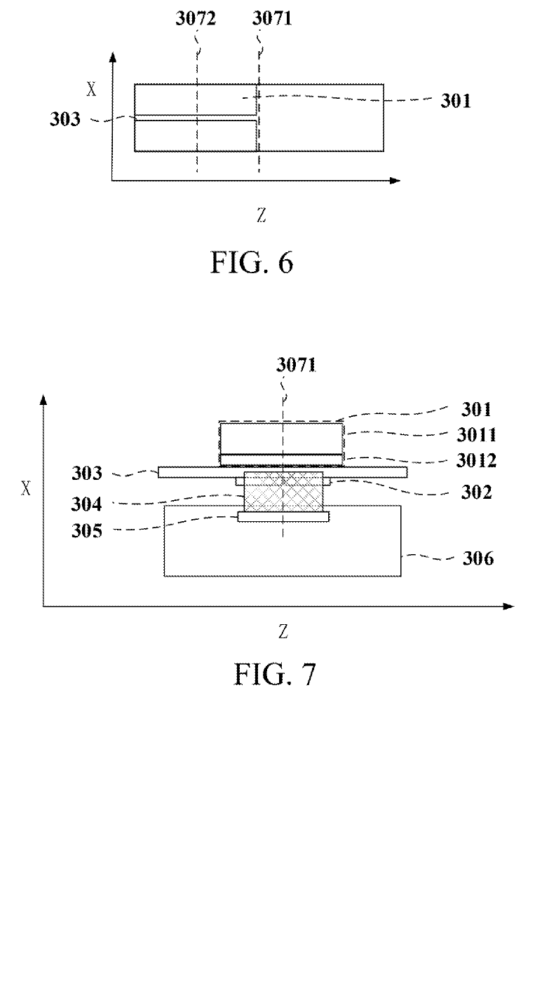

FIG. 5 is a schematic diagram of a cross section of a radiation detector module after a portion of the radiation detector elements in FIG. 3 are removed, viewed along the Y direction. FIG. 6 is a top view of FIG. 5, viewed from FIG. 5 along the X direction. As shown in FIG. 5 and FIG. 6, although the number of rows an acquired image can be reduced by removing half of the radiation detector elements 301 of the radiation detector module along the Z direction, the center of the radiation detector along the Z direction is shifted from the dashed line 3071 to a dashed line 3072, and a shifted distance is, for example, half of the length of the radiation detector element 301 along the Z direction. In FIG. 5 and FIG. 6, the focus position of the X-ray source 103 on the Z axis also requires readjustment to the dashed line 3072, and such adjustment of the focus position of the X-ray source 103 may affect a gantry structure or mechanical vibration of the imaging device, thus affecting imaging quality.

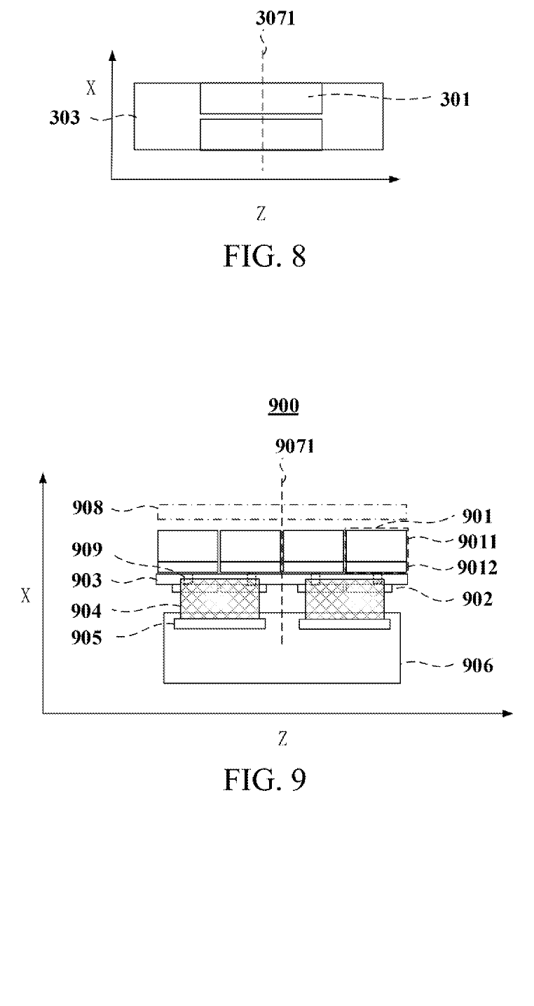

FIG. 7 is another schematic diagram of a cross section of a radiation detector module after a portion of the radiation detector elements in FIG. 3 are removed, viewed along the Y direction. FIG. 8 is a top view of FIG. 7, viewed from FIG. 7 along the X direction. As shown in FIG. 7 and FIG. 8, on the basis of removing half of the radiation detector elements 301 of the radiation detector module along the Z direction, the positions of the remaining radiation detector elements 301 on a surface of the substrate 303 are further adjusted, so that the central position of the remaining radiation detector elements 301 along the Z direction remains at the dashed line 3071. In this way, although no adjustment of the focus position of the X-ray source 103 on the Z axis is required, adjustment of the positions of the remaining radiation detector elements 301 on the surface of the substrate 303 is required. This entails rearrangement or rewiring of the radiation detector elements 301, and even replacement of the circuit substrate 303 or the entire radiation detector module, resulting in increased costs.

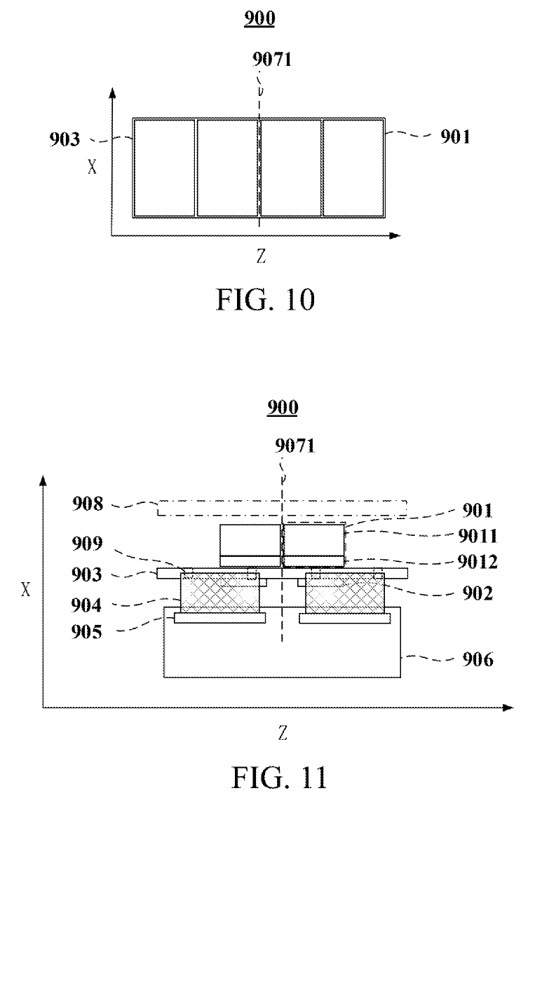

To solve the above problems, or at least a similar problem, an embodiment of the present application provides a radiation detector module. FIG. 9 is a schematic diagram of a cross section of a radiation detector module according to an embodiment of the present application, viewed along the Y direction. FIG. 10 is a schematic top view of a radiation detector module according to an embodiment of the present application, which may be viewed from FIG. 9 along the X direction.

As shown in FIG. 9 and FIG. 10, the radiation detector module 900 of the embodiment of the present application is used to detect a ray signal passing through the subject under examination 105 (as shown in FIG. 1) in an imaging device, and the subject under examination 105 enters or exits the imaging device in the Z direction. The radiation detector module 900 includes a radiation detector element 901, a processing circuit chip 902, and a circuit substrate 903. The radiation detector element 901 receives a ray (for example, an X-ray) emitted by a radiation source (for example, the X-ray source 103 shown in FIG. 1) and converts the ray into an electrical signal.

In some examples, the radiation detector element 901 includes a scintillator 9011 and a photoelectric conversion element 9012. The scintillator 9011 and the photoelectric conversion element 9012 may be correspondingly arranged in a radiation direction of the X-ray, for example, the scintillator 9011 being closer to the radiation source than the photoelectric conversion element 9012. The scintillator 9011 receives a ray emitted by the X-ray source and generates light, such as visible light. The photoelectric conversion element 9012 receives the light generated by the scintillator 9011 and converts the received light into an electrical signal. The photoelectric conversion element 9012 may be a photodiode, for example, a backlit photodiode.

In some other examples, the radiation detector element 901 may not have a scintillator 9011. Therefore, the radiation detector element 901 may directly receive the ray emitted by the radiation source and generate the electrical signal. The radiation detector element 901 may be a photon counting detector, a direct conversion detector, or the like.

A plurality of radiation detector elements 901 are mounted at a first side of the circuit substrate 903 (for example, the side facing the radiation source in the X-direction). In the Z direction of the circuit substrate 903, the plurality of radiation detector elements 901 are arranged in 4N rows, N being an integer greater than or equal to 1. For example, in the example shown in FIG. 10, N=1. To be specific, four radiation detector elements 901 are disposed in the Z direction of the circuit substrate 903.

The processing circuit chip 902 is disposed at a second side of the circuit substrate 903 (for example, the side away from the radiation source in the X direction), and communicates with the radiation detector element 901.

FIG. 13 is another top view of a radiation detector module according to an embodiment of the present application. FIG. 13 shows another arrangement form of a plurality of radiation detector elements 901 on the surface of the circuit substrate 903. As shown in FIG. 13, in the Z direction of the circuit substrate 903, the plurality of radiation detector elements 901 are arranged in 4N+1 rows, N being an integer greater than or equal to 1. For example, in the example shown in FIG. 13, N=1. To be specific, five radiation detector elements 901 are disposed in the Z direction of the circuit substrate 903.

According to the examples of FIG. 9, FIG. 10, and FIG. 13, in the Z direction of the circuit substrate 903, the plurality of radiation detector elements 901 are arranged in 4N rows or 4N+1 rows. In this way, in the case in which a portion of the radiation detector components 901 are removed in the Z direction, the central position of the remaining radiation detector elements in the Z direction can remain unchanged. Therefore, the positions of the remaining radiation detector elements 901 require no adjustment, thereby reducing costs. In addition, the focus position of the radiation source (for example, the X-ray source) requires no adjustment. Thus, no change of a gantry structure of the imaging device is required, and additional mechanical vibration is not caused, thereby ensuring the imaging quality of the imaging device.

As shown in FIG. 9, FIG. 10, and FIG. 13, in the Z direction, the plurality of radiation detector elements 901 are symmetrically arranged with respect to the central position 9071 of the circuit substrate 903 in the Z direction. For example, in the example shown in FIG. 10, the circuit substrate 903 has two radiation detector elements 901 on each of two sides of the central position 9071 in the Z direction. For another example, in the example shown in FIG. 13, the central position 9071 of the circuit substrate 903 in the Z direction coincides with the central position of the radiation detector element 901 arranged in the middle in the Z direction, and there are two radiation detector elements 901 on each of two sides of the radiation detector element 901 arranged in the middle.

Further, in the Z direction, the focus position of the radiation source may be located at the central position 9071 of the circuit substrate 903 in the Z direction.

In the present application, in the radiation detector module 900 as shown in FIG. 9 or FIG. 13, each radiation detector element 901 has M rows of ray transmission channels distributed along the Z direction, and M may be a natural number equal to or greater than 1. In one example, M=16.

In some examples, as shown in FIG. 9, four rows of radiation detector elements 901 are arranged in the Z direction, and the radiation detector module 900 has 16*4=64 rows of channels in the Z direction.

In some examples, as shown in FIG. 13, five rows of radiation detector elements 901 are arranged in the Z direction, and the radiation detector module 900 has 16*5=80 rows of channels in the Z direction.

The radiation detector module 900 shown in FIG. 9 and FIG. 10 can, for example, acquire images of 64 rows of channels, and if it is necessary to adjust the radiation detector module to acquire images of 32 rows of channels, several rows (for example, two rows) of radiation detector elements 901 symmetrically arranged with respect to the central position 9071 may be removed.

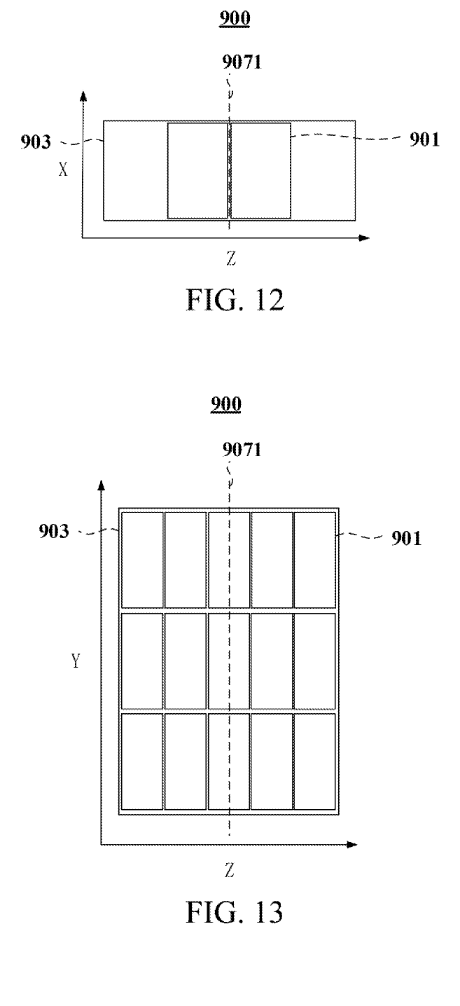

FIG. 11 and FIG. 12 are schematic diagrams of FIG. 9 and FIG. 10 with a portion of the radiation detector elements removed, respectively. As shown in FIG. 11 and FIG. 12, the two outermost rows of radiation detector elements 901 and the corresponding processing circuit chips 902 in FIG. 9 and FIG. 10 may be removed, so that the scanning width of the remaining radiation detector elements 901 of the radiation detector module 900 in the Z direction is reduced by half, the number of channels for acquiring images is reduced by half, and the central position of the remaining radiation detector elements 901 in the Z direction remains unchanged. Therefore, neither the positions of the remaining radiation detector elements 901 nor the focus position of the radiation source (for example, the X-ray source) requires adjustment, thereby saving costs and ensuring the imaging quality of the imaging device.

The radiation detector module 900 shown in FIG. 13 can, for example, acquire images of 80 rows of channels, and if it is necessary to adjust the radiation detector module to acquire images of 48 rows of channels, several rows (for example, two rows) of radiation detector elements 901 symmetrically arranged with respect to the central position 9071 may be removed.

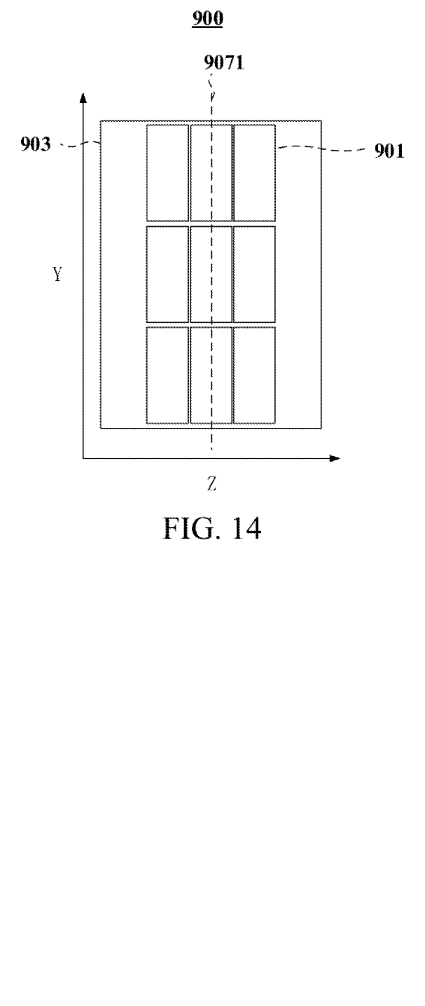

FIG. 14 is a schematic diagram of FIG. 13 with a portion of the radiation detector elements removed. As shown in FIG. 14, the two outermost rows of radiation detector elements 901 and the corresponding processing circuit chips 902 in FIG. 13 may be removed, so that the scanning width of the remaining radiation detector elements 901 of the radiation detector module 900 in the Z direction is reduced by nearly half, for example, the scanning widths of the two radiation detector elements 901 in the Z direction is reduced, and the number of channels for acquiring images is reduced by nearly half, for example, the number of channels for acquiring images of the two radiation detector elements 901 in the Z direction is reduced, and the central position of the remaining radiation detector elements 901 in the Z direction remains unchanged. Therefore, neither the positions of the remaining radiation detector elements 901 nor the focus position of the radiation source (for example, the X-ray source) requires adjustment, thereby saving costs and ensuring the imaging quality of the imaging device.

For the radiation detector module 900 shown in FIG. 13, if it is necessary to further adjust the radiation detector module 900 to acquire images of 16 rows of channels, more rows (for example, four rows) of radiation detector elements 901 arranged symmetrically with respect to the central position 9071 may be removed.

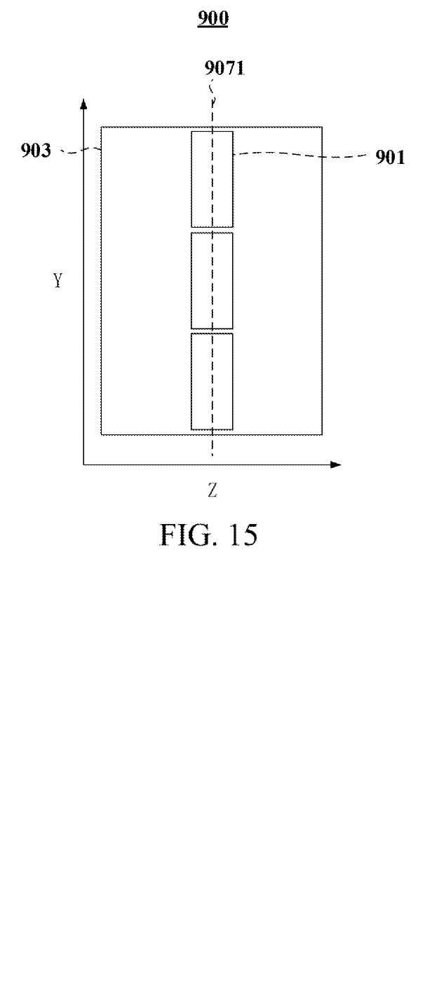

FIG. 15 is another schematic diagram of FIG. 13 with a portion of the radiation detector elements removed. As shown in FIG. 15, the four outermost rows of radiation detector elements 901 and the corresponding processing circuit chips 902 in FIG. 13 may be removed, and a middle row of radiation detector elements 901 and the corresponding processing circuit chips 902 in the Z direction may be retained, so that the scanning width of the remaining radiation detector elements 901 of the radiation detector module 900 in the Z direction and the number of channels for acquiring images are further reduced, and the central position of the remaining radiation detector elements 901 in the Z direction remains unchanged. Therefore, neither the positions of the remaining radiation detector elements 901 nor the focus position of the radiation source (for example, the X-ray source) requires adjustment, thereby saving costs and ensuring the imaging quality of the imaging device.

In the present application, as shown in FIG. 10 or FIG. 13, the plurality of radiation detector elements 901 are arranged in one column (as shown in FIG. 10) or in two or more columns in the Y direction of the circuit substrate 903, and, for example, may be arranged in three columns (as shown in FIG. 13).

In the present application, one (as shown in FIG. 9) or two or more processing circuit chips 902 are disposed in a region of the circuit substrate 903 covered by each radiation detector element 901. In the present application, as shown in FIG. 9 and FIG. 11, the radiation detector element 901 is electrically connected to the corresponding processing circuit chip 902 by means of a conductive path 909 penetrating through the circuit substrate 903.

The radiation detector module further includes a collimator assembly 908. The collimator assembly 908 is disposed on a surface of the radiation detector element 901. The collimator assembly 908 collimates a ray emitted by the radiation source to the radiation detector element 901.

In some examples, rays (for example, X-rays) emitted by the radiation source are collimated by the collimator assembly 908 and then irradiated to the scintillator 9011, light generated by the scintillator 9011 after being irradiated by the rays is converted into an electrical signal by the photoelectric conversion element 9012, and the electrical signal generated by the photoelectric conversion element 9012 is used for tomographic imaging of a subject.

In addition, in some other examples, the radiation detector element 901 may not include the scintillator 9011, so that the rays (for example, X-rays) emitted by the radiation source are collimated by the collimator assembly 908 and then irradiated to the photon counting or direct conversion radiation detector element 901, and the radiation detector element 901 generates an electrical signal.

In the present application, the electrical signal generated by the radiation detector element 901 (for example, the electrical signal generated by the photoelectric conversion element 9012) is transmitted to the processing circuit chip 902 by means of the circuit substrate 903, and the processing circuit chip 902 processes the received electrical signal. For example, the electrical signal received by the processing circuit chip 902 is an analog signal, and the processing circuit chip 902 converts the analog signal into a digital signal, that is, performs analog-digital conversion.

The radiation detector module 900 has a flat panel form factor. The flat panel form factor of the radiation detector module 900 means that the size of a radiation receiving plane of the radiation detector module that receives rays or faces a radiation source is much larger than, or several times the size of, the radiation detector module parallel to a ray propagation path. For example, the length or width of the radiation detector module having a rectangular radiation receiving plane is much greater than the thickness thereof.

The radiation detector module 900 further includes a data collection circuit board 906. The data collection circuit board 906 is electrically connected to the circuit substrate 903 by means of a flexible circuit 904 and a connection circuit 905. A signal (for example, a digital signal) processed by the processing circuit chip 902 is transmitted to the data collection circuit board 906 by the flexible circuit 904 and the connection circuit 905.

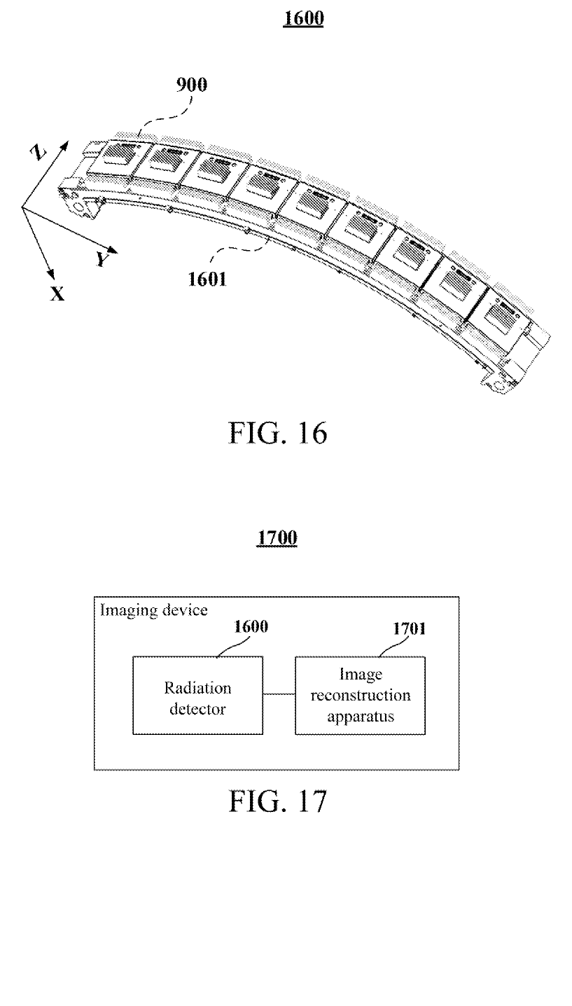

An embodiment of the present application further provides a radiation detector. FIG. 16 is a three-dimensional schematic diagram of a radiation detector according to an embodiment of the present application. As shown in FIG. 16, the radiation detector 1600 includes a guide rail 1601 and two or more radiation detector modules 900 of any one of the foregoing embodiments supported on the guide rail 1601. The radiation detector modules 900 includes 4N or 4N+1 radiation detector elements 301 arranged in the Z direction.

The radiation detector modules 900 are arranged along the Y direction in which the guide rail 1601 extends, and the number of the radiation detector modules 900 ranges from 3 to 15. For example, the radiation detector includes 9 radiation detector modules 900, and the scanning field of view (FOV) of the radiation detector in the Y direction is 50 centimeters (cm). The radiation detector includes 11 radiation detector modules 900, and the scanning field of view (FOV) of the radiation detector in the Y direction is 60 centimeters (cm).

In the radiation detector of the present application, the radiation detector module 900 may have a relatively large area, so that the number of columns of the radiation detector modules 900 can be reduced while still ensuring that the radiation detector reaches a predetermined scanning field of view in the Y direction, and is, for example, one column or two or more columns, thereby reducing the interconnection complexity between the radiation detector modules.

An embodiment of the present application further provides an imaging device. The imaging device is, for example, a medical imaging device, and includes a scanning space for accommodating a subject under examination. The subject under examination enters or exits the scanning space in the Z direction.

FIG. 17 is a schematic diagram of a composition of an imaging device. As shown in FIG. 17, the imaging device 1700 includes the radiation detector 1600 shown in FIG. 16 and an image reconstruction apparatus 1701. The image reconstruction apparatus 1701 performs tomographic imaging of a subject based on an electrical signal generated by the photoelectric conversion element 9012 (as shown in FIG. 9) in a radiation detector module of a radiation detector.

In some examples, the image reconstruction apparatus 1701 may perform image reconstruction by using, for example, data collected by the data collection circuit board 906 in FIG. 9. For the image reconstruction apparatus 1701, reference may be made to the related art. The imaging device of the present application is, for example, a computed tomography (CT) imaging device, a PET-CT, or any other suitable medical imaging device.

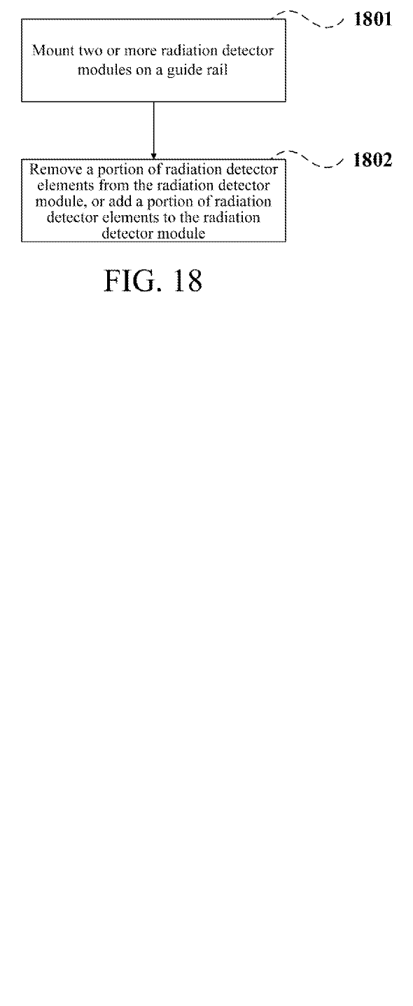

An embodiment of the present application further provides a manufacturing method of a radiation detector. As shown in FIG. 18, the manufacturing method includes mounting two or more radiation detector modules 900 on the guide rail 1601 (Step 1801). In operation 1801, the number of radiation detector modules 900 ranges from, for example, 3 to 15. In operation 1801, the two or more radiation detector modules 900 are arranged along an extension direction of the guide rail 1601, for example, along the Y direction.

In the present application, as shown in FIG. 18, the manufacturing method may further includes removing a portion of the radiation detector elements 901 from the radiation detector module 900, or adding a portion of the radiation detector elements 901 to the radiation detector module 900 (Step 1802). For example, in operation 1802, in the first direction (Z direction), in a symmetrical manner with respect to the central position 9071 of the circuit substrate 903 in the first direction, a portion of the radiation detector elements 901 on two sides of the central position 9071 may be removed, or a predetermined number of radiation detector elements 901 may be mounted on two sides of the central position 9071.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A radiation detector module, used to detect a ray signal passing through a subject under examination in an imaging device, the subject under examination entering or exiting the imaging device in a first direction, wherein the radiation detector module comprises:
a radiation detector element, receiving a ray emitted by a radiation source, and converting the ray into an electrical signal;
a circuit substrate, a plurality of radiation detector elements being mounted at a first side of the circuit substrate, and the plurality of radiation detector elements being arranged in 4N rows or 4N+1 rows in the first direction of the circuit substrate, N being an integer greater than or equal to 1; and
a processing circuit chip, disposed at a second side of the substrate and communicating with the radiation detector elements.

2. The radiation detector module according to claim 1, wherein in the first direction, the plurality of radiation detector elements are symmetrically arranged with respect to the central position of the circuit substrate in the first direction.

3. The radiation detector module according to claim 1, wherein each of the radiation detector elements has 16 rows of ray transmission channels distributed along the first direction.

4. The radiation detector module according to claim 1, wherein the plurality of radiation detector elements are arranged in one column or in two or more columns in a second direction of the circuit substrate.

5. The radiation detector module according to claim 1, wherein at least one of the processing circuit chips is disposed in a region of the circuit substrate covered by each radiation detector element.

6. The radiation detector module according to claim 1, wherein the radiation detector elements are electrically connected to the processing circuit chip by means of a conductive path penetrating through the circuit substrate.

7. The radiation detector module according to claim 1, wherein, the radiation detector element comprises a scintillator and a photoelectric conversion element, the scintillator receiving a ray and generating light, the photoelectric conversion element converting the light generated by the scintillator into an electrical signal, and the photoelectric conversion element comprising a backlit photodiode.

8. The radiation detector module according to claim 1, wherein the radiation detector module has a flat panel form factor.

9. The radiation detector module according to claim 1, wherein the radiation detector module further includes a data collection circuit board, electrically connected to the circuit substrate by means of a wire and receiving data processed by the processing circuit chip.

10. The radiation detector module according to claim 1, wherein the radiation detector module further includes a collimator assembly, disposed on a surface of the radiation detector component, the collimator assembly collimating a ray emitted by the radiation source to the radiation detector element.

11. A radiation detector, wherein the radiation detector comprises a guide rail and two or more radiation detector modules, wherein each radiation detector module includes:
a radiation detector element, receiving a ray emitted by a radiation source, and converting the ray into an electrical signal;
a circuit substrate, a plurality of radiation detector elements being mounted at a first side of the circuit substrate, and the plurality of radiation detector elements being arranged in 4N rows or 4N+1 rows in the first direction of the circuit substrate, N being an integer greater than or equal to 1; and
a processing circuit chip, disposed at a second side of the substrate and communicating with the radiation detector elements.

12. An imaging device, wherein the imaging device comprises a scanning space for accommodating a subject under examination, the subject under examination enters or exits the scanning space in a first direction, the imaging device comprises a radiation detector, and an image reconstruction apparatus, wherein the radiation detector includes
a guide rail and two or more radiation detector modules, wherein each radiation detector module includes:
a radiation detector element, receiving a ray emitted by a radiation source, and converting the ray into an electrical signal;
a circuit substrate, a plurality of radiation detector elements being mounted at a first side of the circuit substrate, and the plurality of radiation detector elements being arranged in 4N rows or 4N+1 rows in the first direction of the circuit substrate, N being an integer greater than or equal to 1; and
a processing circuit chip, disposed at a second side of the substrate and communicating with the radiation detector elements;
and the image reconstruction apparatus performs image reconstruction based on an electrical signal generated by a radiation detector component in a radiation detector module of the radiation detector, so as to generate a tomographic image of the subject under examination.
